(19) United States
(12) Patent Application Publication
(10) Pub. No.: US 2021/0063971 A1
(43) Pub. Date: Mar. 4, 2021

(54) INFORMATION PROCESSING APPARATUS AND NON-TRANSITORY COMPUTER READABLE MEDIUM STORING PROGRAM

(71) Applicant: FUJI XEROX CO., LTD., Tokyo (JP)

(72) Inventors: Masahiro SATO, Kanagawa (JP); Kengo TOKUCHI, Kanagawa (JP)

(73) Assignee: FUJI XEROX CO., LTD., Tokyo (JP)

(21) Appl. No.: 16/740,488

(22) Filed: Jan. 13, 2020

(30) Foreign Application Priority Data
Aug. 26, 2019 (JP) 2019-153630

(51) Int. Cl. G05B 13/02; G06F 3/01

(52) U.S. Cl. CPC G05B 13/021; G06F 3/017; G06F 3/015

(57) ABSTRACT

An information processing apparatus includes a processor configured to operate a device, based on biological information generated from a user who has received an external stimulus.

### CROSS-REFERENCE TO RELATED APPLICATIONS

This application is based on and claims priority under 35 USC 119 from Japanese Patent Application No. 2019-153630 filed Aug. 26, 2019.

### BACKGROUND

### (i) Technical Field

The present invention relates to an information processing apparatus and a non-transitory computer readable medium storing a program.

### (ii) Related Art

It is conceivable to operate a device using biological information such as brain waves.

JP2015-211705A describes an apparatus that detects an electroencephalographic signal from user's brain waves, detects a surface myoelectric biological signal from the user's surface myoelectric potential, and calculates a control signal based on both biological signals.

JP2016-067922A describes a device that acquires a user's brain wave, and selectively operates a plurality of operation devices according to an analysis result obtained by analyzing the brain wave.

### SUMMARY

However, it is not always possible to accurately operate a device based on biological information.

Aspects of non-limiting embodiments of the present disclosure relate to an information processing apparatus and a non-transitory computer readable medium storing a program, capable of improving the accuracy of device operation in a case where the device is operated using biological information.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a processor configured to operate a device, based on biological information generated from a user who has received an external stimulus.

### BRIEF DESCRIPTION OF THE DRAWINGS

Exemplary embodiment(s) of the present invention will be described in detail based on the following figures, wherein:

FIG. 1 is a block diagram illustrating a configuration of an information processing system according to the present exemplary embodiment;

FIG. 2 is a block diagram illustrating a configuration of an information processing apparatus according to the present exemplary embodiment;

FIG. 3 is a diagram illustrating a management table; and

FIG. 4 is a diagram illustrating a management table.

### DETAILED DESCRIPTION

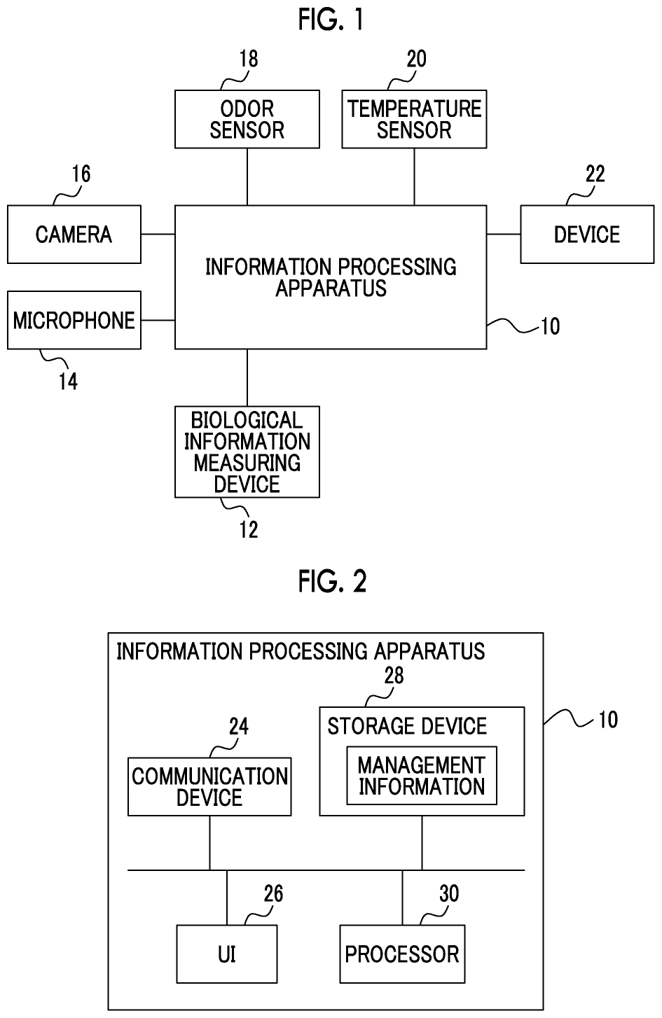

An information processing system according to the present exemplary embodiment will be described with reference to FIG. 1. FIG. 1 illustrates an example of the configuration of the information processing system according to the present exemplary embodiment.

The information processing system according to the present exemplary embodiment includes an information processing apparatus 10, one or a plurality of biological information measuring devices 12, one or a plurality of microphones 14, one or a plurality of cameras 16, and one or a plurality of odor sensors 18, one or a plurality of temperature sensors 20, and one or a plurality of devices 22. The number of devices and sensors shown in FIG. 1 is only an example, and the number of devices and sensors is not limited to the number of devices and sensors shown in FIG. 1. The information processing system may include other devices (for example, external devices such as servers) other than the device shown in FIG. 1.

The information processing apparatus 10, the biological information measuring device 12, the microphone 14, the camera 16, the odor sensor 18, the temperature sensor 20, and the device 22 are configured to communicate with other devices, other sensors, and the like. The communication may be wired communication using a cable, or wireless communication. That is, each device and each sensor may be physically connected to another device, another sensor, or the like by a cable to transmit and receive information to and from each other, or may transmit and receive information to and from each other by wireless communication. For example, near field communication, Wi-Fi (registered trademark), or the like is used as the wireless communication. Wireless communication of standards other than these may be used. Near field communication is, for example, Bluetooth (registered trademark), radio frequency identifier (RFID), NFC, or the like. Each device may communicate with another device, another sensor, or the like through a communication path such as a local area network (LAN) or the Internet.

The information processing apparatus 10 is, for example, a personal computer (hereinafter referred to as “PC”), a tablet PC, a smartphone, a mobile phone, or other apparatuses. The information processing apparatus 10 may be a terminal apparatus (for example, a tablet PC, a smartphone, a mobile phone, or the like) that can be carried by the user, or may be a device that is installed on a table or the like and used. The information processing apparatus 10 may be a smart speaker having a communication function, a microphone, and a speaker. The information processing apparatus 10 may be installed indoors (for example, a room floor, a ceiling, a table, or the like) or may be installed outdoors. Further, the information processing apparatus 10 may be a movable apparatus (for example, a self-propelled apparatus).

The biological information measuring device 12 includes a sensor, an electrode, and the like, and is configured to measure a user's biological information. In a case where the plurality of biological information measuring devices 12 are included in the information processing system, biological information measuring devices 12 may measure different types of biological information, respectively. Of course, a part or all of the biological information measuring devices 12 may be configured to measure the same type of biological information. Each biological information measuring device 12 may be configured to measure one type of biological information or may be configured to measure a plurality of types of biological information.

The biological information measuring device 12 transmits the biological information measured by the own device to the information processing apparatus 10. The biological information measuring device 12 may transmit the biological information to the information processing apparatus 10 every time the biological information is measured, or store the biological information and transmit the biological information to the information processing apparatus 10 at predetermined time intervals, or transmit the biological information to the information processing apparatus 10 at a timing designated by the user. The biological information measuring device 12 may receive the biological information measured by another type of biological information measuring device 12 from the other biological information measuring device 12, and transmit the biological information measured by the own device and the biological information measured by the other type of biological information measuring device 12 to the information processing apparatus 10.

The biological information measuring device 12 may analyze the biological information measured by the own device or the other type of biological information measuring device, and transmit information indicating the analysis result to the information processing apparatus 10. For example, the biological information measuring device 12 may include a processor, and the processor may analyze the biological information. Of course, the analysis may be performed by the information processing apparatus 10.

The biological information measuring device 12 includes a battery, and may be driven by power supplied from the battery, or may be driven by receiving power supplied from the information processing apparatus 10.

The biological information measuring device 12 may be a wearable device that measures biological information by the entire biological information measuring device 12 being worn on a user. For example, the biological information measuring device 12 may be a device worn on the user's head, a bearable device worn on the user's ear, or a device worn on the user's arm, hand, wrist, or finger (for example, a wristwatch-type device), a device worn around the user's neck, or a device worn on the user's body or legs.

The biological information is various types of physiological information and anatomical information emitted from a user who is a living body. The category of the concept of biological information includes, for example, information indicating brain activity (for example, brain wave, cerebral blood flow, cerebral magnetic field signal, or the like), pulse rate, blood pressure, heart rate, electrocardiographic waveform, electromyographic waveform, eye movement, body temperature, sweating, line of sight, voice, user's movement and the like. These are only examples of biological information, and other types of physiological information or anatomical information may be used as the biological information. The biological information measuring device 12 may measure one piece of biological information among these pieces of biological information, or may measure a plurality of pieces of biological information.

The information processing apparatus 10 receives biological information from the biological information measuring device 12, and analyzes the biological information, stores the biological information, outputs the biological information, stores information indicating the analysis result of the biological information, and outputs information indicating the analysis result of the biological information. Of course, analysis of biological information may be performed by the biological information measuring device 12. Outputting the biological information includes, for example, displaying the biological information, outputting the biological information as voice information, and the like. Outputting information indicating the analysis result of the biological information includes, for example, displaying information indicating the analysis result, outputting the analysis result as voice information, and the like. The information processing apparatus 10 may transmit biological information and information indicating the analysis result to another apparatus.

The information processing apparatus 10 may include one or a plurality of biological information measuring devices 12. That is, one or a plurality of biological information measuring devices 12 may be incorporated into the information processing apparatus 10 to constitute one device. The entire information processing apparatus 10 including one or a plurality of biological information measuring devices 12 may be worn by a user to measure biological information. That is, the information processing apparatus 10 may be a wearable apparatus. For example, the information processing apparatus 10 may be a device worn on the user's head, a bearable device worn on the user's ear, or a device worn on the user's arm, hand, wrist, or finger (for example, a wristwatch-type device), a device worn around the user's neck, or a device worn on the user's body or legs.

Of course, the information processing apparatus 10 and the biological information measuring device 12 may be separate devices. For example, the information processing apparatus 10 may be a smart speaker, and the biological information measuring device 12 may be a wearable device worn by a user.

The microphone 14 is a device that collects sound waves. Sound around the microphone 14 (for example, human voice or other sounds) is input to the microphone 14, and sound data is generated by the microphone 14. The sound data representing the sound input to the microphone 14 corresponds to an example of environment information indicating the environment around the microphone 14. In that sense, the microphone 14 corresponds to an example of an environment information measuring device. The sound data representing the user's voice input to the microphone 14 corresponds to an example of the user's biological information. In that sense, the microphone 14 corresponds to an example of a biological information measuring device that measures user's biological information.

The camera 16 is an imaging device. The surroundings of the information processing apparatus 10 and other places are captured by the camera 16, and image data representing the surroundings and image data representing other places are generated. The image data may be moving image data or still image data. The image data captured by the camera 16 corresponds to an example of environment information indicating an environment included in the capturing range of the camera 16. In that sense, the camera 16 corresponds to an example of an environment information measuring device. Further, the image data representing the user, generated by capturing the user with the camera 16, corresponds to an example of the user's biological information. For example, the movement of the user, the body shape of the user, and the like detected from the image data correspond to an example of the user's biological information. In that sense, the camera 16 corresponds to an example of a biological information measuring device that measures user's biological information.

The odor sensor 18 is a device that measures the odor around the odor sensor 18 and generates odor data representing the measured odor. The odor data representing the odor measured by the odor sensor 18 corresponds to an example of environment information indicating the environment around the odor sensor 18. In that sense, the odor sensor 18 corresponds to an example of an environment information measuring device. Further, the odor data representing the odor of the user measured by the odor sensor 18 corresponds to an example of the user's biological information. In that sense, the odor sensor 18 corresponds to an example of a biological information measuring device that measures user's biological information.

The temperature sensor 20 is a device that measures the temperature around the temperature sensor 20 and generates temperature data representing the measured temperature. The temperature data representing the temperature measured by the temperature sensor 20 corresponds to an example of environment information indicating the environment around the temperature sensor 20. In that sense, the temperature sensor 20 corresponds to an example of an environment information measuring device. Further, the temperature data representing the temperature of the user measured by the temperature sensor corresponds to an example of the user's biological information. In that sense, the temperature sensor 20 corresponds to an example of a biological information measuring device that measures user's biological information.

In addition, the camera 16, the odor sensor 18, and the temperature sensor 20 may not be included in the information processing system, and at least one may be included in the information processing system. Other environment information measuring devices (for example, a humidity sensor, an illuminance sensor, an atmospheric pressure sensor, an infrared sensor, or the like) may be included in the information processing system. One or a plurality of environment information measuring devices may be included in the information processing apparatus 10. For example, the camera 16 may be included in the information processing apparatus 10. For example, the microphone 14 may be included in the information processing apparatus 10.

The device 22 is, for example, a PC, a tablet PC, a smartphone, a mobile phone, a robot (for example, a humanoid robot, a non-human animal robot, a cleaning robot, and other robots), a projector, a display device such as a liquid crystal display, a recording device, a playback device, an imaging device such as a camera, a refrigerator, a rice cooker, a microwave oven, a coffee maker, a vacuum cleaner, a washing machine, an air conditioner, a lighting device, a watch, a surveillance camera, an automobile, a motorcycle, aircraft (for example, unmanned aircraft (for example, drone)), a game machine, a gas range, a warm water washing toilet seat, a ventilation fan, a doorbell, an entrance monitor, an elevator, a door, a window, or various sensing devices (for example, a temperature sensor, a humidity sensor, a voltage sensor, a current sensor, or the like). The category of the concept of the device 22 may include general devices. For example, information device, video device, audio device, and other device may also be included in the category of the device 22 according to the present exemplary embodiment.

Hereinafter, the configuration of the information processing apparatus 10 will be described in detail.

The information processing apparatus 10 includes, for example, a communication device 24, a UI 26, a storage device 28, and a processor 30. The information processing apparatus 10 may include other configurations.

The communication device 24 is a communication interface, and has a function of transmitting data to other apparatuses and a function of receiving data transmitted from other apparatuses. The communication device 24 may have a wireless communication function or may have a wired communication function. The communication device 24 may communicate with other devices by using, for example, near field communication, or may communicate with other devices through a communication path such as a LAN or the Internet. The communication device 24 communicates with the biological information measuring device 12, and receives the biological information transmitted from the biological information measuring device 12. The communication device 24 may transmit control information for controlling the operation of the biological information measuring device 12 to the biological information measuring device 12. Further, the communication device 24 communicates with the environment information measuring device to receive the environment information transmitted from the environment information measuring device. The communication device 24 may transmit control information for controlling the operation of the environment information measuring device to the environment information measuring device.

The UI 26 is a user interface, and includes a display device and an operation device. The display device is a liquid crystal display, an EL display, or the like. The operation device is a keyboard, input keys, an operation panel, or the like. The UI 26 may be a UI such as a touch panel that has both a display device and an operation device. In addition, a microphone may be included in the UI 26, and a speaker that emits sound may be included in the UI 26.

The storage device 28 is a device that constitutes one or a plurality of storage areas for storing various types of data. The storage device 28 is, for example, a hard disk drive, various memories (for example, RAM, DRAM, ROM, or the like), other storage devices (for example, an optical disk), or a combination thereof. One or a plurality of storage devices 28 are included in the information processing apparatus 10.

The storage device 28 stores management information. The management information is information for managing biological information for operating the device 22. For example, for each operation of the device 22, control information indicating the operation of the device 22 and reference biological information that is assumed to be generated from a user who performs the operation when the device 22 is operated are associated in advance, and registered in the management information. It can be said that the reference biological information is information indicating the operation of the device 22. For each user, control information and reference biological information may be associated with each other and registered in the management information.

The processor 30 is configured to operate the device 22, based on biological information generated from a user who has received an external stimulus.

The external stimulus is, for example, a stimulus related to the operation of the device 22. For example, an environment where the operation of the device 22 is required corresponds to an example of an external stimulus. The environment in which the operation of the device 22 is necessary is, for example, a conversation related to the device 22, and an odor or a temperature in which the operation of the device 22 is necessary. The processor 30 detects an external stimulus based on the environment information measured by the environment information measuring device, and determines whether or not the user has received the external stimulus. Further, the processor 30 determines whether or not the biological information measured from the user is biological information generated from the user who has received an external stimulus. That is, the processor 30 determines whether or not the biological information measured from the user is biological information generated from the user due to an external stimulus. In a case where the biological information measured from the user is the biological information generated from the user who has received an external stimulus, that is, the biological information measured from the user is biological information generated from the user due to the external stimulus, the processor 30 operates the device 22 based on the biological information. The environment information measuring device is, for example, a microphone 14, a camera 16, an odor sensor 18, a temperature sensor 20, and the like. Sensors other than these may be used as the environment information measuring device. The processor 30 determines whether or not the user has received an external stimulus, based on, for example, at least one of sound data measured by the microphone 14, image data captured by the camera 16, odor data measured by the odor sensor 18, or temperature data measured by the temperature sensor 20. The processor 30 may determine whether the user has received an external stimulus based on data other than these.

For example, the user and the surroundings thereof are captured by the camera 16, and the processor 30 analyzes the image data generated by the capturing to determine whether or not the user has received an external stimulus. To explain with a specific example, in a case where the image data shows the user's hand being pinched, the processor 30 recognizes that the biological information (for example, brain wave) measured at that time is the biological information generated from the user due to an external stimulus of a user's hand being pinched.

For example, specific environment information is measured, and the biological information measured by the user after the specific environment information is measured is estimated to be biological information generated from the user who has received an external stimulus. That is, the measured biological information is estimated to be biological information generated from the user due to an external stimulus. In this case, the environment represented by the specific environment information corresponds to an example of an external stimulus. For example, the biological information measured from the user until a predetermined time elapses from the time when the specific environment information is measured is estimated to be the biological information generated from the user who has received an external stimulus. The environment represented by the specific environment information is, for example, an environment related to the operation of the device 22. For example, in a case where specific environment information related to the operation of the device 22 is measured, the processor 30 operates the device 22 based on the biological information measured by the user after the specific environment information is measured. By operating the device 22 based on the biological information generated from the user due to external stimulus, the user's intention can be reflected and the operation accuracy of the device 22 can be improved.

For example, in a case where the difference between the biological information generated from the user and measured by the biological information measuring device 12 and the reference biological information registered in the management information is within an allowable range, the processor 30 transmits control information associated with the reference biological information to the device 22, and the device 22 is operated. The allowable range is predetermined, for example. The allowable range may be changed by the user.

For example, the processor 30 determines whether or not the difference between the waveform represented by the biological information measured by the user and the waveform represented by the reference biological information is within an allowable range. As an element for the determination, for example, a waveform feature such as a shape, a peak, a period, or an amplitude of waveform is used.

For example, a brain machine interface may be constructed by the biological information measuring device 12 that measures brain activity and the information processing apparatus 10. The brain machine interface method may be invasive or non-invasive. In this case, the processor 30 operates the device 22 based on the user's brain activity (for example, brain waves). In order to operate the device 22, the processor 30 may extract a characteristic component from the brain wave, and operate the device 22 based on the extracted component. In order to extract characteristic components from the brain wave, Fast Fourier Transform (FFT), Wavelet Transform (WT), Time Frequency Distribution (TFD), Eigenvector Methods (EM), autoregressive model (ARM) or the like may be used. In addition, as a method for linking the brain wave and the operation of the device 22 using the feature vector obtained by the feature extraction, for example, independent component analysis (ICA), k-average method, support vector machine (SVM), a convolutional neural network, or the like may be used.

The processor 30 is configured to control the operation of each unit of the information processing apparatus 10. The processor 30 may include a memory.

Hereinafter, the operation of the information processing system according to the present exemplary embodiment will be described with a specific example.

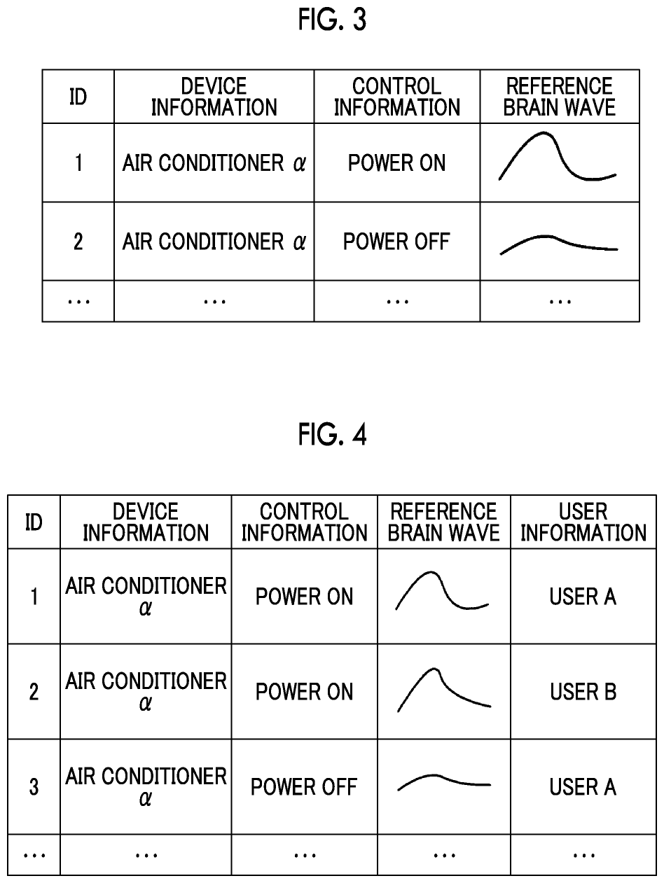

FIG. 3 shows an example of a management table that is an example of management information. Data of the management table is stored in the storage device 28. The data of the management table may be stored in an external device such as a server without being stored in the storage device 28.

In the management table, for each operation of the device 22, an ID, device information, control information, and a reference brain wave that is an example of reference biological information are associated with each other in advance. The management table shown in FIG. 3 is not a management table reflecting specific biological information of individual users. Here, brain waves are used as an example of biological information, but biological information other than brain waves may be used.

The ID is information for managing the operation of the device 22. The device information is information for identifying the device 22 to be operated, and is, for example, the name, device ID, or model number of the device 22. The control information is information indicating the operation of the device 22. The reference brain wave is biological information indicating the operation of the device 22 and is assumed to be generated from a user who performs the operation when the device 22 is operated. The reference brain wave is, for example, a brain wave that is determined by statistical processing and is assumed to be generally generated from a user who performs an operation associated with the reference brain wave. In addition, the information indicating the operation specified by analyzing the brain wave may be registered in the management table without the brain wave itself (that is, the waveform) being registered in the management table.

Hereinafter, a specific example will be described.

The information associated with the ID “1” is information regarding the operation of “air conditioner α”, which is an example of the device 22.

The control information associated with the ID “1” is information indicating an operation of “turning on the power when the power of “air conditioner α” is off”.

The reference brain wave associated with the ID “1” is a brain wave indicating an operation of “turning on the power of “air conditioner α””. That is, this reference brain wave is a brain wave that is assumed to be emitted from the user when the user turns on the power in a case where the power of the “air conditioner α” is off.

For example, when the power of “air conditioner α” is off, in a case where a reference brain wave associated with ID “1” is measured from the user due to an external stimulus, the processor 30 turns on the power of the “air conditioner α” according to the control information associated with the ID “1”. For example, the processor 30 transmits the control information associated with the ID “1” to the “air conditioner α”, thereby turning on the power of the “air conditioner α”.

When the power of “air conditioner α” is off, in a case where a brain wave whose difference from the reference brain wave associated with ID “1” is within an allowable range due to an external stimulus is measured from the user, the processor 30 may turn on the power of the “air conditioner α” according to the control information associated with the ID “1”. For example, the processor 30 extracts features such as the shape, peak, period, and amplitude of the brain wave from each of the brain wave measured from the user and the reference brain wave, compares the features of waveforms, and determines whether or not a difference between the features of both waveforms is within the allowable range.

Further, the processor 30 may calculate the similarity between the brain wave measured from the user and the reference brain wave, and may determine whether or not the similarity is equal to or greater than a threshold. The threshold is a value corresponding to the allowable range. In a case where the similarity between the two waveforms is equal to or greater than the threshold, the processor 30 determines that the two waveforms are similar, and determines that the difference between the waveform measured from the user and the reference brain wave is within an allowable range. In other words, when the power of “air conditioner α” is off, in a case where a brain wave whose similarity with the reference brain wave associated with the ID “1” is equal to or greater than the threshold is measured from the user, the processor 30 may turn on the power of the “air conditioner α” according to the control information associated with the ID “1”.

For operations other than the operation of ID “1”, similarly to the operation of ID “1”, the device 22 is operated based on the user's brain wave generated due to external stimulus.

Note that a combination of a plurality of different reference biological information and control information may be associated with each other, for each operation of the device 22. For example, as the reference biological information, the brain wave that is assumed to be emitted from the user when the user turns on the power in a state where the power of the “air conditioner α” is off (that is, the reference brain wave) and the body temperature that the user is assumed to have at that time (that is, the reference body temperature) may be associated with the control information indicating the operation of turning on the power of the “air conditioner α”. In this case, in a case where the difference between the brain wave measured from the user due to an external stimulus and the reference brain wave is within the allowable range, and the difference between the user's body temperature measured at that time and the reference body temperature is within the allowable range, the processor 30 turns on the power of the “air conditioner α” according to the control information associated with the reference brain wave and the reference body temperature. Biological information other than brain wave and body temperature may be used.

Note that when the user operates the device 22 based on the biological information, the user emits a voice representing device information for identifying the device 22 to be operated, and the processor 30 may specify the device 22 to be operated by analyzing the voice. Note that the user's voice is measured by the microphone 14. The processor 30 searches the management table for the device 22 to be operated, and controls the operation on the device 22. For example, in a state where a brain wave indicating an operation of turning on the power of “air conditioner α” is emitted from the user, in a case where the user utters a voice representing “air conditioner α”, the processor 30 specifies, based on the voice, the device 22 to be operated as “air conditioner α”, and turns on the power of “air conditioner α” according to the control information associated with the measured brain wave. In the example illustrated in FIG. 3, the operation associated with the ID “1” and the operation associated with the ID “2” are registered in the management table for the operation for “air conditioner α”. The processor 30 compares the brain wave associated with each of ID “1” and ID “2” with the brain wave measured from the user. In a case where the difference between the brain wave associated with ID “1” and the brain wave measured from the user is within an allowable range, and the difference between the brain wave associated with ID “2” and the brain wave measured from the user is not within the allowable range, the processor 30 operates the “air conditioner α” according to the control information associated with the ID “1”. In this case, the processor 30 turns on the power of “air conditioner α”. On the other hand, in a case where the difference between the brain wave associated with ID “1” and the brain wave measured from the user is not within an allowable range, and the difference between the brain wave associated with ID “2” and the brain wave measured from the user is within the allowable range, the processor 30 operates the “air conditioner α” according to the control information associated with the ID “2”. In this case, the processor 30 turns off the power of “air conditioner α”.

In addition, for each user, device information, control information, and reference biological information may be associated with each other and registered in the management table. Specifically, specific reference biological information of each user may be registered in the management table.

FIG. 4 shows a management table in which specific reference biological information of each user is registered. In the management table illustrated in FIG. 4, for each operation of the device 22, an ID, device information, control information, a reference brain wave that is an example of reference biological information, and user information are associated are with each other.

User information is information (for example, a user name, a user ID, or the like) for identifying a user. Note that the user information may not be registered in the management table.

The reference brain wave associated with the user information is a brain wave measured from the user when the user indicated by the user information performs an operation indicated by the control information associated with the reference brain wave. The reference brain wave of each user is measured in advance.

For example, when the user A manually turns on the power of the air conditioner α, the brain wave of the user A is measured by the biological information measuring device 12, and the measured brain wave is registered in the management table as a reference brain wave representing an operation that the user A turns on the power of the air conditioner α. In this case, device information indicating “air conditioner α”, control information indicating an operation of turning on the power of “air conditioner α”, the measured reference brain wave of the user A, and user information for identifying user A are associated with each other and registered in the management table. The registration work may be performed using the information processing apparatus 10 or may be performed using another apparatus. In the example shown in FIG. 4, these pieces of information are registered as information of ID “1”. The same applies to other operations and other users.

The registration work may be performed a plurality of times, and an average of a plurality of brain waves measured thereby may be registered as a reference brain wave. For example, a work of measuring the brain wave generated from the user A by the biological information measuring device 12 when the user A manually turns on the power of the air conditioner α is performed a plurality of times, and the average of a plurality of brain waves measured by this work may be registered in the management table as a reference brain wave.

For example, when the power of “air conditioner α” is off, in a case where a brain wave whose difference from the reference brain wave associated with ID “1” is within an allowable range due to an external stimulus is measured, the processor 30 turns on the power of the “air conditioner α” according to the control information associated with the ID “1”.

For example, in a state where only the user A logs in to the information processing apparatus 10, in a case where a brain wave having the difference from the reference brain wave associated with the ID “1” being within an allowable range is measured from the user A, the processor 30 turns on the power of “air conditioner α” according to the control information associated with the ID “1”. Specifically, when only the user A is logged in to the information processing apparatus 10, in a case where the brain wave is measured by the biological information measuring device 12, the processor 30 searches for the reference brain wave associated with the user information for identifying the user A who logs in and registered in the management table. In the example illustrated in FIG. 4, since the reference brain wave associated with ID “1” and the reference brain wave associated with ID “3” are registered in the management table as the reference brain wave of user A, these reference brain waves are searched. In a case where the measured brain wave matches the reference brain wave associated with ID “1”, or the difference between the measured brain wave and the reference brain wave associated with ID “1” is within an allowable range, the processor 30 turns on the power of the “air conditioner α” according to the control information associated with the ID “1”. In a case where the measured brain wave matches the reference brain wave associated with ID “3”, or the difference between the measured brain wave and the reference brain wave associated with ID “3” is within an allowable range, the processor 30 turns off the power of the “air conditioner α” according to the control information associated with the ID “3”.

As another example, in a state where the user operating the device 22 is set as “user A” in the information processing apparatus 10, in a case where the reference brain wave associated with the ID “1” is measured from the user A, the processor 30 may turn on the power of “air conditioner α” according to the control information associated with the ID “1”. Specifically, in a case where a brain wave is measured by the biological information measuring device 12 in a state where “user A” is set as a user operating the device 22 in the information processing apparatus 10, the processor 30 searches for the reference brain wave associated with the user information for identifying the user A who is operating the device 22 and registered in the management table. In a case where the measured brain wave matches the reference brain wave associated with ID “1”, or the difference between the measured brain wave and the reference brain wave associated with ID “1” is within an allowable range, the processor 30 turns on the power of the “air conditioner α” according to the control information associated with the ID “1”. A user who operates the device 22 is set in the information processing apparatus 10 by the user, for example.

As for the user other than the user A, as with the user A, each information is registered in the management table. For example, each piece of information associated with the ID “2” is information regarding an operation when the user B turns on the power of “air conditioner α”. Each piece of information associated with the ID “3” is information regarding an operation when the user A turns off the power of “air conditioner α”.

The processor 30 outputs information for inquiring the user whether or not to operate the device 22, before operating the device 22 based on the biological information, and may operate the device 22 in a case of receiving an instruction to operate the device 22 from the user. Outputting information for inquiring the user whether or not to operate the device 22 may be, for example, displaying an image representing the content of the operation on the display unit of the UI 26 or emitting a voice representing the content of the operation from a speaker. That is, the processor 30 may prompt the user to determine whether or not the operation is possible using an image, voice, or the like. In a case where an agreement to execute the operation is obtained from the user, the processor 30 operates the device 22 by transmitting control information associated with the reference brain wave in which the difference from the brain wave measured from the user is included in the allowable range to the device 22. For example, in a case where a brain wave representing agreement is measured from the user, the processor 30 operates the device 22 by transmitting the control information to the device 22.

In a case where the operation indicated by the control information associated with the reference brain wave whose difference from the brain wave measured from the user is included in the allowable range is an operation for stopping the operation of the device 22, the processor 30 may stop the operation of the device 22, without inquiring the user whether or not to operate the device 22. For example, in a case where the operation indicated by the control information associated with the reference brain wave whose difference from the brain wave measured from the user is within the allowable range is an operation to turn off the power of the device 22 or an operation for temporarily stopping the operation of the device 22, the processor 30 turns off the power of the device 22 or temporarily stops the operation of the device 22, without inquiring the user whether or not to operate the device 22.

In a case where the operation associated with the reference brain wave whose difference from the brain wave measured from the user is within the allowable range is an operation to lower the performance level of the device 22, the processor 30 may lower the performance level of the device 22, without inquiring the user whether or not to operate the device 22. Reducing the performance level of the device 22 means reducing the output level of the device 22. For example, lowering the volume of the speaker, reducing the air volume of the air conditioner, raising the temperature of the cooling, lowering the temperature of the heating, or the like are examples of lowering the performance level.

While the user is sleeping, the processor 30 may stop the operation of the device 22 based on the biological information. The processor 30 determines whether or not the user is sleeping, based on biological information measured from the user, by using, for example, a known technique. It is known that a person's sleep state can be recognized based on the type, intensity, transition, and the like of a brain wave. Sleep states are classified into, for example, REM sleep, non-REM sleep, and awakening. Of course, this classification is only an example, and another classification may be used. Here, as an example, it is assumed that in a case where the user's sleep state is REM sleep or non-REM sleep, the user's state is a sleeping state, and in a case where the user's sleep state is awakening, the user's state is not sleeping. The processor 30 determines the sleep state of the user based on the type, intensity, transition, and the like of the measured brain wave, by using a known technique.

It is known that brain waves when falling asleep are generated in the order of β wave, α wave, θ wave, and δ wave. In a state where the user lies down and closes his or her eyes, the β wave is generated. From that state, the body relaxes, and the α wave is generated. From that stage, θ waves are generated in a case where consciousness fades, and δ waves are generated in a case where sleep is deep. The processor 30 determines the sleep state of the user by analyzing the brain wave generated in this way.

An acceleration sensor which is an example of the biological information measuring device 12 may be worn by the user, and measure the user's acceleration, that is, movement. The processor 30 may recognize the sleep state of the user, based on the magnitude of the acceleration and the amount of change. Further, the processor 30 may recognize the sleep state of the user, based on a user's pulse rate, blood pressure, or the like.

In a case where the device 22 to be operated is not present within the operation range, the processor 30 may output a warning. For example, the processor 30 may display information indicating a warning on the display unit of the UI 26 or may emit a warning sound. The operation range is, for example, a range within a predetermined distance from the position on the basis of the predetermined position. The predetermined position may be a user position, a position where the information processing apparatus 10 is installed, or any position designated by the user. For example, the position of the user is specified by specifying the position of a terminal device (for example, a smartphone) carried by the user by global positioning system (GPS) or the like. The position of the information processing apparatus 10 may be set in the information processing apparatus 10, or may be specified by GPS or the like.

The processor 30 may operate each device 22 by transmitting control information corresponding to the biological information to each device 22 in a case of receiving the biological information indicating an operation instruction for each of the plurality of devices 22.

The processor 30 may change the performance level of the device 22 according to the magnitude of the change in the biological information measured from the user. For example, for each device 22, information indicating the magnitude of the change in biological information and information indicating the performance level are associated with each other and stored in the storage device 28. The processor 30 changes the performance level according to the change of the biological information by referring to the associated information. For example, the processor 30 changes the performance level of the device 22 according to the magnitude of the change in the amplitude of the brain wave, which is an example of biological information. Specifically, the processor 30 improves the performance level as the change in the amplitude of the brain wave increases. For example, the processor 30 increases the volume of the speaker, increases the air volume of the air conditioner, lowers the cooling temperature, or raises the heating temperature, as the change in the amplitude of the brain wave increases. As another example, the processor 30 may change the performance level of the device 22 according to the magnitude of the change in the peak of the brain wave, the magnitude of the change in the period, or the like.

The processor 30 may change the performance level of the device 22 according to the change speed of the biological information measured from the user. For example, for each device 22, information indicating the speed of the change in biological information and information indicating the performance level are associated with each other and stored in the storage device 28. The processor 30 changes the performance level according to the change speed of the biological information by referring to the associated information. For example, the processor 30 changes the performance level of the device 22 according to the change speed of the amplitude of the brain wave, which is an example of biological information. Specifically, the processor 30 improves the performance level as the amplitude of the brain wave changes fast. For example, the processor 30 increases the volume of the speaker, increases the air volume of the air conditioner, lowers the cooling temperature, or raises the heating temperature, as the amplitude of the brain wave changes fast. As another example, the processor 30 may change the performance level of the device 22 according to the speed of change in the peak of the brain wave, the speed of change in the period, or the like.

In a case where a plurality of pieces of biological information are measured from the user, the processor 30 may operate the device 22 based on the biological information according to a predetermined priority. For example, in a case where a plurality of pieces of biological information are simultaneously measured from the user, the processor 30 operates the device 22 based on the biological information according to the priority. For example, in a case where each of the plurality of pieces of biological information indicates an operation on the identical device 22, the processor 30 operates the identical device 22 based on the biological information, according to the priority determined based on the content of the operation. As another example, the priority may be determined based on the type of biological information. For example, the priority of a brain wave is set to the highest priority, and the priority of pulse is set to the second highest priority. Priorities are also set in advance for other types of biological information. For example, in a case where a brain wave and a pulse are measured, the processor 30 operates the device 22 based on the brain wave with a higher priority, and does not operate the device 22 based on the pulse with a lower priority. Even in a case where another type of biological information is measured, the processor 30 operates the device 22 based on the biological information having a higher priority than the other biological information.

In a case where the user's state is different from the predetermined normal state, the processor 30 may stop the operation of the information processing apparatus 10. The processor 30 determines the state of the user based on the biological information measured from the user. For example, in a case where the difference between the user's body temperature and the body temperature in the normal state (for example, normal heat) is equal to or greater than the threshold, the processor 30 determines that the user's state is different from the normal state, and stops the operation of the information processing apparatus 10. Thereby, operation of the device 22 based on biological information is no longer performed. The processor 30 may determine whether or not the user's state is a normal state based on the user's pulse and blood pressure.

In a case where, within a predetermined time from the time when the biological information indicating the operation (hereinafter referred to as “first operation”) on the device 22 is measured from the user, another type of biological information indicating an operation (hereinafter referred to as “second operation”) different from the operation is measured from the user, the processor 30 operates the device 22, based on the biological information according to a predetermined priority. The second operation is an operation on the device 22 on which the first operation is performed. For example, the priority may be determined based on the content of the operation. In a case where the content of the first operation has a higher priority than the content of the second operation, the processor 30 performs the first operation on the device 22. As another example, the priority may be determined based on the measurement order of biological information. For example, the priority of biological information measured later is higher than the biological information measured previously. In this case, the processor 30 performs a second operation on the device 22. The priorities described above are merely examples, and the priorities may be determined by the user.

Hereinafter, specific modification examples will be described in detail.

### Modification Example 1

In Modification Example 1, the specific voice measured by the microphone 14 corresponds to an example of an external stimulus, and the voice data representing the specific voice corresponds to an example of specific environment information. Further, biological information is a brain wave as an example. The specific voice is, for example, a voice related to the operation of the device 22 (for example, conversation). The biological information measured from the user after the voice data representing the specific voice is measured is estimated to be the biological information generated from the user who has received the specific voice that is an external stimulus. In this case, the processor 30 operates the device 22 based on the biological information generated from the user who has received a specific voice. In this case, the processor 30 operates the device 22 based on the biological information generated from the user who has heard a specific voice. For example, the biological information measured from the user until a predetermined time elapses from the time when the specific voice is measured is estimated to be the biological information generated from the user who has heard the specific voice that is an external stimulus. In other words, the measured biological information is estimated to be biological information generated from the user due to hearing a specific voice that is an external stimulus.

For example, the voice of the conversation partner of the user (for example, user A) whose brain wave, which is biological information, is measured corresponds to an example of an external stimulus, and voice data representing the voice corresponds to an example of specific environment information.

In a case where the user A whose brain wave is measured and the conversation partner have a conversation, the processor 30 receives the brain wave measured from the user A who has heard the voice of the conversation partner, and the operation associated with the brain wave is specified. For example, the processor 30 refers to the management table shown in FIG. 4 and specifies the operation associated with the brain wave measured from the user A. Specifically, the processor 30 specifies an operation associated with a reference brain wave in which a difference from the brain wave measured from the user A is included in an allowable range. The processor 30 may display an image representing the content of the operation on the display unit of the UI 26 or may emit a voice representing the content of the operation from a speaker. In a case where an agreement to execute the operation is obtained from the user A, the processor 30 operates the device 22 by transmitting control information associated with the reference brain wave in which the difference from the brain wave measured from the user A is included in the allowable range to the device 22.

For example, voice data representing the voice of the user A and the conversation partner is measured by the microphone 14, and the processor 30 recognizes the content of the conversation between the user A and the conversation partner by analyzing the voice data measured by the microphone 14. Further, the user A and the conversation partner may be captured by the camera 16, and the processor 30 may recognize the user A and the conversation partner based on the image data generated by the capturing. The processor 30 recognizes the content of the speech of the conversation partner related to the operation of the device 22, receives the brain wave measured from the user A by listening to the speech, and transmits control information associated with the reference brain wave having a difference from the brain wave of the user A being within an allowable range to the device 22. The content of the conversation partner's speech regarding the operation of the device 22 corresponds to an example of an external stimulus, and the voice data representing the content of the speech corresponds to an example of specific environment information. A brain wave measured after the conversation partner's speech (for example, a brain wave measured until a predetermined time elapses from the time when the conversation partner speaks) is estimated to be the brain wave emitted from the user A who has heard the conversation partner's speech, that is, a brain wave emitted from the user A who has received an external stimulus.

A specific example will be described. In a case where the conversation partner says, “Do you want to see OO on TV?”, the content of the speech is measured by the microphone 14 and the processor 30 recognizes the content of the speech by analyzing the voice measured by the microphone 14. The content of the speech is a speech related to the operation of the television (TV), which is the device 22, and corresponds to an example of an external stimulus, and the voice data representing the content of the speech corresponds to an example of specific environment information. The processor 30 receives a brain wave measured from the user A who has received the speech. For example, a brain wave measured until a predetermined time elapses from the time when the conversation partner says, “Do you want to watch OO on TV?” is estimated to be a brain wave emitted from the user A who has heard the conversation partner's speech. In a case where the brain wave is a brain wave representing consent, the processor 30 outputs a message with the content “Do you want to turn on the TV?”. For example, the processor 30 may emit the voice of the message from a speaker, or may display the message on the display unit of the UI 26. The processor 30 receives a brain wave measured from the user A after the message is output. In a case where the brain wave is a brain wave representing consent, the processor 30 transmits control information for executing an operation of turning on the television that is the device 22 to the television, thereby turning on the power of the television.

### Modification Example 2

In Modification Example 2, the specific odor measured by the odor sensor 18 corresponds to an example of an external stimulus, and odor data representing the specific odor corresponds to an example of specific environment information. For example, the odor sensor 18 measures the odor around the user. Further, biological information is a brain wave as an example.

The specific odor is, for example, an odor related to the operation of the device 22. The biological information measured from the user after the odor data representing the specific odor is measured is estimated to be the biological information generated from the user who has received the specific odor that is an external stimulus. In this case, the processor 30 operates the device 22 based on the biological information generated from the user who has received a specific odor. In this case, the processor 30 operates the device 22 based on the biological information generated from the user who has smelled a specific odor. For example, the biological information measured from the user until a predetermined time elapses from the time when the specific odor is measured is estimated to be the biological information generated from the user who has smelled the specific odor which is an external stimulus. That is, the measured biological information is estimated to be biological information generated from the user due to smelling a specific odor that is an external stimulus.

Ina case where a specific odor is measured, the processor 30 receives a brain wave measured from the user who has smelled the specific odor, and specifies an operation associated with the brain wave. For example, the processor 30 refers to the management table shown in FIG. 4 and specifies the operation associated with the brain wave measured from the user. Specifically, the processor 30 specifies an operation associated with a reference brain wave in which a difference from the brain wave measured from the user is included in an allowable range. The processor 30 may display an image representing the content of the operation on the display unit of the UI 26 or may emit a voice representing the content of the operation from a speaker. In a case where an agreement to execute the operation is obtained from the user, the processor 30 operates the device 22 by transmitting control information associated with the reference brain wave in which the difference from the brain wave measured from the user is included in the allowable range to the device 22. A brain wave measured after a specific odor related to the operation of the device 22 is measured (for example, a brain wave measured until a predetermined time elapses from the time when the specific odor is measured) is estimated to be the brain wave emitted from the user who has smelled the specific odor, that is, a brain wave emitted from the user who has received an external stimulus.

A specific example will be described. After cooking, the odor during cooking remains in the room, and the odor sensor 18 measures odor data representing the odor. The odor is an odor related to the operation of the air purifier, which is the device 22, and corresponds to an example of an external stimulus, and odor data representing the odor corresponds to an example of specific environment information. The processor 30 receives a brain wave measured from the user who has smelled the odor. For example, a brain wave representing “odor is uncomfortable”, which is measured until a predetermined time elapses from the time when the odor after cooking is measured is estimated to be a brain wave emitted from the user who has smelled the odor. In this case, the processor 30 outputs a message with the content “Do you want to turn on the air purifier?”. For example, the processor 30 may emit the voice of the message from a speaker, or may display the message on the display unit of the UI 26. The processor 30 receives a brain wave measured from the user after the message is output. In a case where the brain wave is a brain wave representing consent, the processor 30 transmits control information for executing an operation of turning on the air purifier that is the device 22 to the air purifier, thereby turning on the power of the air purifier. In addition, an aroma diffuser for diffusing a scent may be used with an air purifier or instead of an air purifier.

### Modification Example 3

In Modification Example 3, the temperature included in the specific temperature range, measured by the temperature sensor 20, corresponds to an example of an external stimulus, and the temperature data indicating the temperature included in the specific temperature range corresponds to an example of specific environment information. For example, the temperature sensor 20 measures the temperature around the user. Further, biological information is a brain wave as an example. For example, the temperature included in the specific temperature range is a temperature related to the operation of the device 22. The biological information measured by the user after the temperature data representing the temperature included in the specific temperature range is measured is estimated to be biological information generated from the user who has received a temperature that is an external stimulus. In this case, the processor 30 operates the device 22, based on the biological information generated from the user who has received the temperature included in the specific temperature range. That is, the processor 30 operates the device 22, based on the biological information generated from the user who has felt the temperature included in the specific temperature range. For example, the biological information measured from the user until a predetermined time elapses from the time when the temperature included in the specific temperature range is measured is estimated to be the biological information generated from the user who has felt the temperature included in the specific temperature range which is an external stimulus. In other words, the measured biological information is estimated to be biological information generated from the user due to feeling a temperature included in a specific temperature range that is an external stimulus.

In a case where the temperature included in the specific temperature range is measured, the processor 30 receives the brain wave measured from the user who has felt the temperature included in the specific temperature range, and specifies the operation associated with the brain wave. For example, the processor 30 refers to the management table shown in FIG. 4 and specifies the operation associated with the brain wave measured from the user. Specifically, the processor 30 specifies an operation associated with a reference brain wave in which a difference from the brain wave measured from the user is included in an allowable range. The processor 30 may display an image representing the content of the operation on the display unit of the UI 26 or may emit a voice representing the content of the operation from a speaker. In a case where an agreement to execute the operation is obtained from the user, the processor 30 operates the device 22 by transmitting to the device 22, control information associated with the reference brain wave in which the difference from the brain wave measured from the user is included in the allowable range. A brain wave measured after the temperature related to the operation of the device 22 is measured (for example, a brain wave measured until a predetermined time elapses from the time when the temperature included in the specific temperature range is measured) is estimated to be the brain wave emitted from the user who has felt the temperature, that is, a brain wave emitted from the user who has received an external stimulus.

A specific example will be described. It is assumed that when the user returns home, the room is hot and the temperature sensor 20 installed in the room has measured a temperature equal to or greater than a predetermined threshold. The temperature equal to or greater than the predetermined threshold is a temperature included in a specific temperature range. Here, as an example, the temperature is generally estimated that a person feels hot. The temperature included in the specific temperature range is a temperature related to the operation of the air conditioner that is the device 22, and corresponds to an example of an external stimulus, and the temperature data representing the temperature corresponds to an example of specific environment information. The processor 30 receives a brain wave measured from the user who has felt the temperature. For example, a brain wave representing “hot and uncomfortable”, which is measured until a predetermined time elapses from the time when the temperature included in the specific temperature range is measured is estimated to be a brain wave emitted from the user who has felt the temperature. In this case, the processor 30 outputs a message with the content “Do you want to turn on the air conditioner?”. For example, the processor 30 may emit the voice of the message from a speaker, or may display the message on the display unit of the UI 26. The processor 30 receives a brain wave measured from the user after the message is output. In a case where the brain wave is a brain wave representing consent, the processor 30 transmits control information for executing an operation of turning on the air conditioner that is the device 22 to the air conditioner, thereby turning on the power of the air conditioner.

### Modification Example 4

In Modification Example 4, the specific situation viewed by the user whose biological information is measured corresponds to an example of an external stimulus, and image data representing the specific situation corresponds to an example of specific environment information. For example, the user and the surroundings thereof are captured by the camera 16. Further, biological information is a brain wave as an example. The specific situation is, for example, a situation related to the operation of the device 22. The biological information measured from the user after the image data representing the specific situation is measured is estimated to be the biological information generated from the user who has received the specific situation that is an external stimulus. In this case, the processor 30 operates the device 22 based on the biological information generated from the user who has received a specific situation. That is, the processor 30 operates the device 22 based on the biological information generated from the user who has viewed a specific situation. For example, the biological information measured from the user until a predetermined time elapses from the time when the image data representing the specific situation is captured is measured is estimated to be the biological information generated from the user who has viewed the specific situation that is an external stimulus. That is, the measured biological information is estimated to be biological information generated from the user because the user views a specific situation that is an external stimulus.

For example, the processor 30 analyzes the image data representing the user and the surroundings thereof, specifies the direction at which the user looks, and recognizes the situation that the user views.

In a case where image data representing a specific situation is captured, the processor 30 receives a brain wave measured from a user who has viewed the specific situation, and specifies an operation associated with the brain wave. For example, the processor 30 refers to the management table shown in FIG. 4 and specifies the operation associated with the brain wave measured from the user. Specifically, the processor 30 specifies an operation associated with a reference brain wave in which a difference from the brain wave measured from the user is included in an allowable range. The processor 30 may display an image representing the content of the operation on the display unit of the UI 26 or may emit a voice representing the content of the operation from a speaker. In a case where an agreement to execute the operation is obtained from the user, the processor 30 operates the device 22 by transmitting to the device 22, control information associated with the reference brain wave in which the difference from the brain wave measured from the user is included in the allowable range. A brain wave measured after the image data representing the specific situation related to the operation of the device 22 is captured (for example, a brain wave measured until a predetermined time elapses from the time when the image data representing the specific situation is captured) is estimated to be the brain wave emitted from the user who has viewed the specific situation, that is, a brain wave emitted from the user who has received an external stimulus.

A specific example will be described. It is assumed that the user finds the cotton dust falling on the floor, the camera 16 captures an image of the user watching the situation, and image data representing the situation is generated. The situation where dust is falling on the floor is a situation related to the operation of the robot cleaner, which is the device 22, and corresponds to an example of an external stimulus, and image data representing the situation corresponds to an example of specific environment information. The processor 30 receives a brain wave measured from the user who has seen the situation. For example, a brain wave representing “dust is uncomfortable”, which is measured until a predetermined time elapses from the time when image data representing the appearance of the user looking at cotton dust falling on the floor is captured is estimated to be a brain wave emitted from the user who has seen the situation. In this case, the processor 30 outputs a message with the content “Do you want to turn on the robot cleaner?”. For example, the processor 30 may emit the voice of the message from a speaker, or may display the message on the display unit of the UI 26. The processor 30 receives a brain wave measured from the user after the message is output. In a case where the brain wave is a brain wave representing consent, the processor 30 transmits control information for executing an operation of turning on the robot cleaner that is the device 22 to the robot cleaner, thereby turning on the power of the robot cleaner.

In the embodiments above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device). In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing apparatus comprising:
a processor configured to operate a device, based on biological information generated from a user who has received an external stimulus.

2. The information processing apparatus according to claim 1,
wherein in a case where a difference between the biological information and reference biological information indicating an operation of the device is within an allowable range, the processor operates the device based on the biological information.

3. The information processing apparatus according to claim 1,
wherein before operating the device based on the biological information, the processor outputs information for inquiring the user whether or not to operate the device, and in a case of receiving an instruction to operate the device from the user, the processor operates the device.

4. The information processing apparatus according to claim 2,
wherein before operating the device based on the biological information, the processor outputs information for inquiring the user whether or not to operate the device, and in a case of receiving an instruction to operate the device from the user, the processor operates the device.

5. The information processing apparatus according to claim 3,
wherein in a case of stopping the operation of the device, the processor stops the operation of the device, without inquiring the user whether or not to the operate the device.

6. The information processing apparatus according to claim 4,
wherein in a case of stopping the operation of the device, the processor stops the operation of the device, without inquiring the user whether or not to operate the device.

7. The information processing according to claim 3,
wherein in a case of reducing a performance level of the device, the processor reduces the performance level of the device, without inquiring the user whether or not to operate the device.

8. The information processing apparatus according to claim 4,
wherein in a case of reducing a performance level of the device, the processor reduces the performance level of the device, without inquiring the user whether or not to operate the device.

9. The information processing apparatus according to claim 1,
wherein the external stimulus is a voice of a conversation partner of the user.

10. The information processing apparatus according to claim 2,
wherein the external stimulus is a voice of a conversation partner of the user.

11. The information processing apparatus according to claim 1,
wherein the external stimulus is an odor.

12. The information processing apparatus according to claim 1,
wherein the external stimulus is a temperature.

13. The information processing apparatus according to claim 1,
wherein the external stimulus is a situation that the user views.

14. The information processing apparatus according to claim 1,
wherein the processor does not operate the device based on the biological information, while the user is sleeping.

15. The information processing apparatus according to claim 1,
wherein the processor changes a performance level of the device according to a magnitude of a change in the biological information.

16. The information processing apparatus according to claim 1,
wherein the processor changes a performance level of the device according to a change speed of the biological information.

17. The information processing apparatus according to claim 1,
wherein in a case where a plurality of pieces of biological information are measured from the user, the processor operates the device, based on the biological information, according to a predetermined priority.

18. The information processing apparatus according to claim 1,
wherein in a case where, within a predetermined time from a time when the biological information indicating the operation on the device is measured from the user, another type of biological information indicating an operation different from the operation is measured from the user, the processor operates the device, based on the biological information, according to a predetermined priority.

19. The information processing apparatus according to claim 1,
wherein the biological information is a brain wave.

20. A non-transitory computer readable medium storing a program causing a computer to operate a device, based on biological information generated from a user who has received an external stimulus.
